(19) United States
(12) Patent Application Publication
(10) Pub. No.: US 2012/0083841 A1
(43) Pub. Date: Apr. 5, 2012

(54) SUTURE ANCHOR WITH SUTURE MANAGEMENT

(75) Inventors: Kristian DiMatteo, Raynham, MA (US); Gregory R. Whittaker, Raynham, MA (US); Nathan Cauldwell, Raynham, MA (US)

(73) Assignee: DePuy Mitek, Inc., Raynham, MA (US)

(21) Appl. No.: 13/241,781

(22) Filed: Sep. 23, 2011

Related U.S. Application Data
(60) Provisional application No. 61/388,028, filed on Sep. 30, 2010.

(51) Int. Cl. A61B 17/04

(52) U.S. Cl. 606/232

(57) ABSTRACT

A suture anchor comprises an anchor body comprising a longitudinal axis and bone engaging external threads oriented for rotation about the longitudinal axis; and a suture attachment on the anchor body rotational with respect to the body about the longitudinal axis whereby to help reduce twisting of one or more sutures which may be attached to the suture attachment as the anchor body is threaded into a bone.

### RELATED APPLICATIONS

This application claims the priority benefit of U.S. Provisional Application No. 61/388,028, filed Sep. 30, 2010, the entire contents of which are hereby incorporated by reference.

### BACKGROUND

This invention relates to suture anchors for attaching suture to bone, and more specifically to such suture anchors which employ a suture management feature.

It is known to use suture anchors for attaching soft tissues such as ligaments or tendons to adjacent bone. Threaded suture anchors employ external threads and are rotated into the bone, usually into a pre-drilled bone hole, to affix the suture anchor to the bone. They will typically employ an eyelet or other feature for affixing one or more sutures to the anchor. Managing the sutures extending from the suture anchor can be a challenge.

### SUMMARY OF THE INVENTION

A suture anchor according to the present invention comprises an anchor body having a longitudinal axis and bone engaging external threads oriented for rotation about the longitudinal axis. A suture attachment on the anchor body is rotational with respect to the body about the longitudinal axis whereby to help reduce twisting of one or more sutures which may be attached to the suture attachment as the anchor body is threaded into a bone.

Preferably, the body and suture attachment are provided sterile and packaged in a bacteria proof enclosure. One or more sutures can be attached to the suture attachment.

Preferably, a loop of suture is affixed to the suture attachment, the loop having ends extending outwardly from the suture anchor body and suture attachment. Also preferably, a plurality of suture loops of suture are affixed to the suture attachment, each loop having ends extending outwardly from the suture anchor body and suture attachment.

Preferably, an axial bore enters the body from a proximal end thereof with the suture attachment located on the body such that one or more sutures attached thereto can extend out of the axial bore at the body proximal end. Preferably, the body comprises a first abutment surface within the axial bore and the suture attachment comprises an opposing second abutment surface engaged with the first abutment surface such that the suture attachment is rotatable in the axial bore against the first abutment surface with the engagement between the first abutment surface and the second abutment surface preventing the suture attachment from moving proximally out of the axial bore. Preferably, the first abutment surface and second abutment surface are annular.

In one aspect of the invention, the suture attachment comprises a disc having a plurality of axial holes therethrough for receiving one or more loops of suture therethrough.

In one aspect of the invention, the suture attachment comprises a proximal cylindrical portion having one or more radially extending bosses thereon, the cylindrical portion being received within the axial bore with the one or more bosses adjacent a wall of the anchor body forming the axial bore such that suture looped about the one or more bosses is retained by the one or more bosses within the body between the wall and cylindrical portion.

A method according to the present invention provides for attaching tissue to a bone. The method comprises the steps of: passing a first portion of a suture length through the tissue; threading a suture anchor having a second portion of the suture length into a bone hole in the bone, the suture anchor comprising an anchor body comprising a longitudinal axis and bone engaging external threads oriented for rotation about the longitudinal axis; and a suture attachment on the anchor body rotational with respect to the body about the longitudinal axis with the second portion of the suture length attached to the suture attachment; and rotating the suture attachment with respect to the anchor body to reduce twisting of the suture length induced by the step of threading the suture anchor body into the bone hole.

Preferably, the second suture portion comprises a loop of suture affixed to the suture attachment and a pair of suture segments extending therefrom and the step of rotating the suture attachment reduces twisting of the pair of suture segment with respect to each other.

### BRIEF DESCRIPTION OF THE DRAWINGS

FIG. 1 is an exploded perspective view of a suture anchor according to the present invention;

FIG. 2 is a perspective view of a suture receiver of the suture anchor of FIG. 1;

FIG. 3 is a perspective view of the suture receiver of FIG. 2 with an alternative method for engaging multiple suture loops;

FIG. 4 is a cut-away view of the suture anchor of FIG. 1 being implanted into a bone;

FIG. 5 is an exploded perspective view of a further embodiment of a suture anchor according to the present invention;

FIG. 6 is an exploded perspective view of a further embodiment of a suture anchor according to the present invention;

FIG. 7A is a perspective view of a further embodiment of a suture anchor according to the present invention;

FIG. 7B is an exploded perspective view of the distal portion of the suture anchor of FIG. 7A; and

FIG. 7C is a cut-away view of the distal portion of the suture anchor of FIG. 7A.

### DETAILED DESCRIPTION

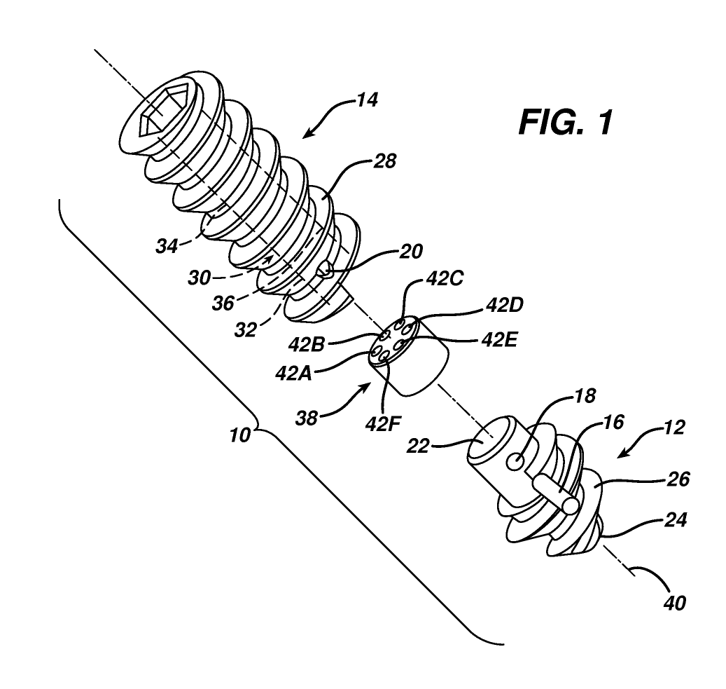

FIG. 1 illustrates a suture anchor 10 according to the present invention. Suture anchor 10 comprises a distal body portion 12 which connects to a proximal body portion 14 both of which are held together by a pin 16 through respective cross-bores 18 and 20 in the distal body portion 12 and the proximal body portion 14. The distal body portion 12 is generally cylindrical in shape having a proximal end 22 through which the cross-bore 18 passes and a distal end 24 which tapers inwardly for easier insertion of the suture anchor 10 into a bone hole (not shown in FIG. 1). An external screw thread 26 encircles the distal body portion 12.

The proximal body portion 14 is also generally cylindrical in shape and bears an external screw thread 28 which mates with the thread 26 to form a continuous threading about the suture anchor 10. An axial-bore 30 passes through the proximal body portion 14. A distal portion 32 thereof, has a larger diameter than a proximal portion 34 thereby creating an internal annual abutment 36. The proximal portion 34 has a hexagonal cross-section to accommodate a driving tool (not shown in FIG. 1). One with ordinary skill in the art would appreciate that alternative tool/anchor engagements may be employed.

The bore-distal portion 32 has a circular cross section and is sized to closely accommodate the body distal portion proximal end 22. A suture receiver 38 fits within the axial-bore distal portion 32 between the abutment 36 and the distal body portion proximal end 22. It has a circular cross-section and is sized to rotate freely about a longitudinal axis 40 of the suture anchor 10. Six axial suture bores 42A-F pass through the suture receiver 38.

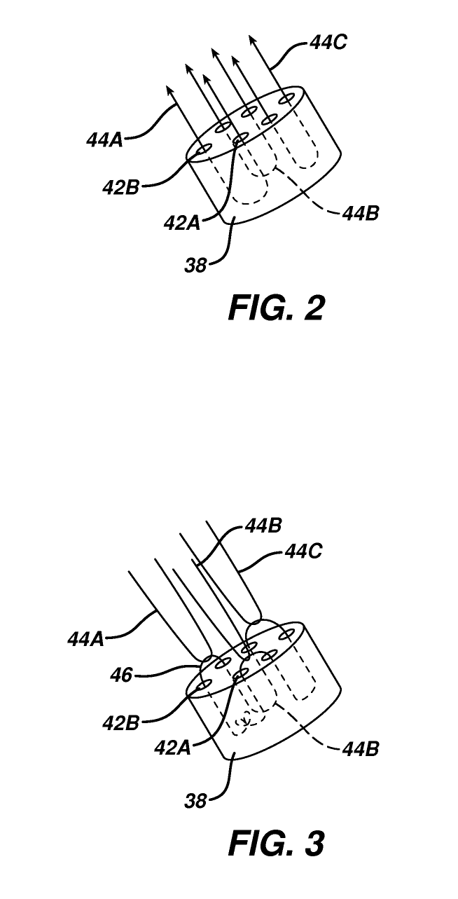

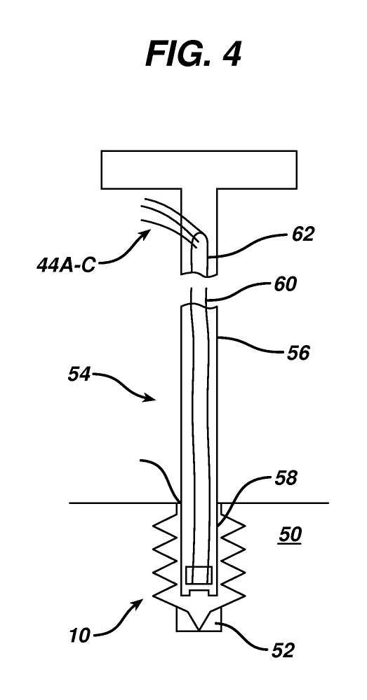

Turning also now to FIG. 2, the suture bores 42A-F accommodates three suture loops 44A-C. Alternatively, as shown in FIG. 3, a suture strand 46 can be woven through the suture bores 42A-F such that the suture loops 44A-C can be threaded through portions of the suture strand 46 extending above the suture receiver 38. Ends 48 of the each of the suture loops 44A-C extend proximally out of the suture anchor 10 through the axial bore 30. The suture ends 48 can then be attached to soft tissue as is understood by one of skill in the art for attaching that soft tissue to a bone. For instance, in a rotator cuff repair, each of the suture loops 44A-C may extend through the rotator cuff tendon at different locations to effect a repair.

The design of the suture anchor 10 provides advantages both in manufacturability and in performance. Having the suture loops 44A-C attach internal of the suture anchor and extending out through the axial bore 30, provides a preferred angle of exit toward soft tissue versus a proximal attachment point. With the separate distal body portion 12 and proximal body portion 14 attaching the suture loops 44A-C deep within the suture anchor 10 becomes easier during manufacturing. When inserting a conventional threaded anchor, any suture loops extending therefrom tend to twist due to the twisting of the anchor in its insertion. The present suture receiver 38 is free to rotate within the axial bore 30 thus reducing twisting of the suture loops 44A-C as the suture anchor 10 is inserted.

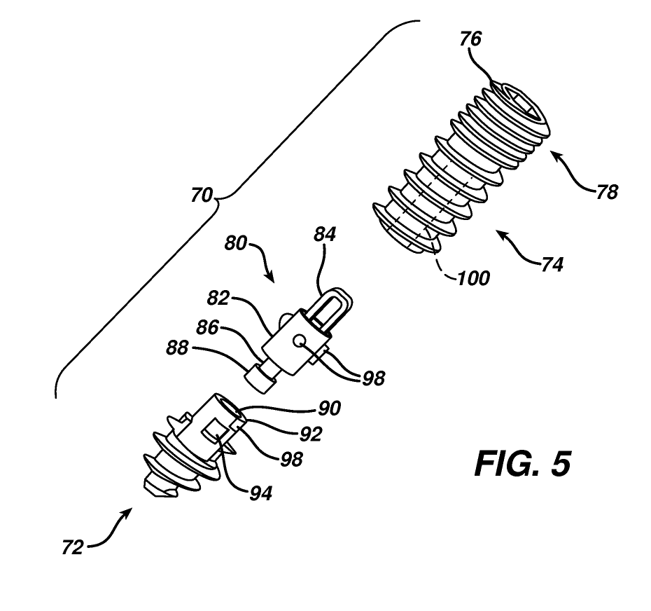

Turning also now to FIG. 4, preferably, the suture anchor 10 is inserted into a bone 50 having a pre-drilled hole 52. A driver 54 having an elongated body 56 and an external hex shape at its distal tip 58, mates with the hex shaped axial bore proximal portion 34. The suture loops 44A-C extend proximally out of the suture anchor 10 through a canulation 60 through the driver distal tip 58 and body 56. In one preferred embodiment, the canulation 60 is open 62 along one side much that after the suture anchor is driven into the bone hole 52 by the driver 54, the driver 54 can be removed from the suture anchor 10 with the suture loops 44A-C disengaging from the driver 54 through the canulation opening 62.

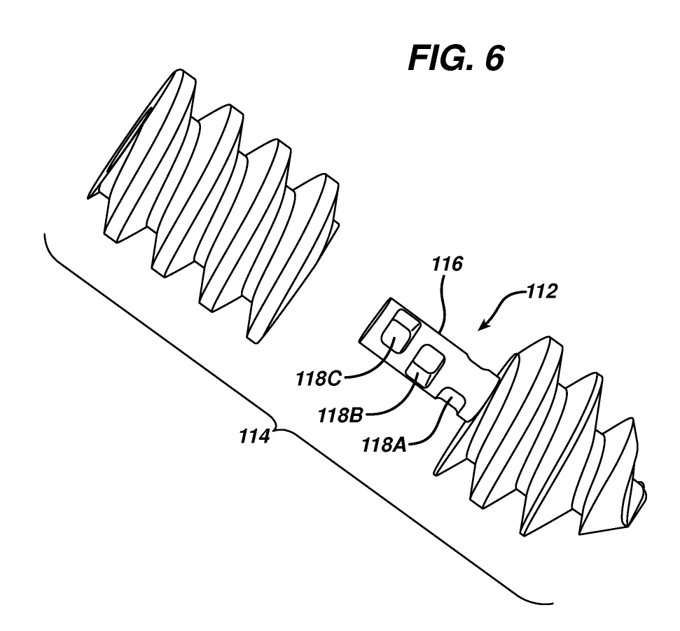

FIG. 5 illustrates and alternative embodiment of a suture anchor 70 comprising a distal threaded body portion 72 and proximal threaded body portion 74. The suture anchor 70 is similar in most respects to the suture anchor 10. However, the proximal body portion 74 carries an additional thread start 76 at its proximal portion 78 thereby effectively increasing thread pitch at the proximal portion 78 and thus enhancing purchase within the hard cortical bone (not shown in FIG. 5) with which this portion typically engages. Also, the anchor 70 employs a somewhat different suture receiver 80.

The suture receiver 80 comprises a central cylindrical body 82 having a proximally extending suture eyelet 84. A distally extending post 86 terminates in an annular flange 88. The anchor distal body portion 72 has an axial bore 90 having a distal body portion 92 sized to accommodate the flange 88 and a narrower proximal portion 94 sized to accommodate the post 86 with the diameter differences creating an internal annular abutment 96 against which the flange 88 bears preventing its proximal movement out of the bore 90. A lateral cut out 98 is provided in the distal body portion 72 to allow the flange 88 and post 86 to be inserted laterally into distal bore 90. This arrangement allows the suture receiver 80 to rotate freely within the distal bore (90).

Several bosses 98 extend outwardly radially from the suture receiver body 82 and fit closely, yet with rotation, within an axial bore 100 in the proximal body portion 74. Suture loops (not shown in FIG. 5) can be passed through the eyelet 84 and also, if desired, around each of the bosses 98, and then proximally out of the anchor through the bore 100, the loops about the bosses being held by the bosses 98 and not being able to slip between the bosses 98 and the body 74 due to the close fit of the bosses 98 within the bore 100.

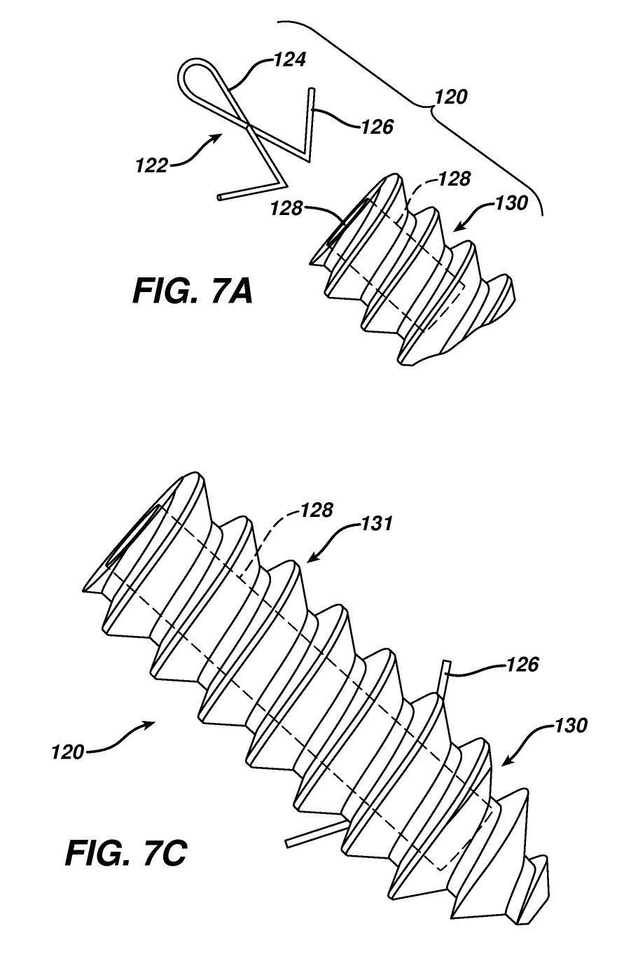

FIG. 6 illustrates a further embodiment of a suture anchor 110 similar to that shown in FIG. 5. A suture receiver 112 is rotatably received within a distal body portion 114 as in the previous embodiment however its proximal end 116 is structured differently. It is cylindrical and bears three suture eyelets 118 A-C which are spaced apart both axially and rotationally, with the middle eyelet 118 B passing laterally through the proximal end 116 at 45 degrees with respect to the top eyelet 118 A and bottom eyelet 118 C and with the top and bottom eyelets 118 A and 118 C passing laterally through the proximal end and being oriented normal to each other. The placement and orientation of the eyelets 118 A-C assists in suture management, both in separation and in identification of individual loops.

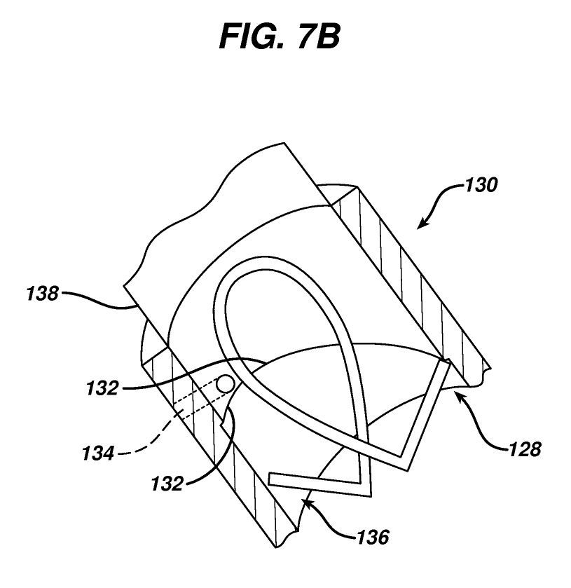

FIGS. 7 A-C. illustrate a further suture anchor 120 having an alternative suture receiver 122 in the form of a bent loop 124 of spring metal having proximally extending ends 126. It is received within an axial bore 128 of a distal body portion 130 of the anchor 120 (which attaches to a proximal body portion 131). One or more lengths of suture (not shown in FIGS. 7 A-C) can attach to the loop 124. Proximal force therefrom draws the ends 126 up along a camming ledge 132 within the bore 128 toward angled side ports 134 from the bore 128 through the distal body portion 130, allowing the ends 126 to protrude from the distal body portion 130 (see FIG. 7 C) and provide additional fixation within a bone (not shown in FIGS. 7 A-C). While the anchor 120 is being inserted a distal end 136 of a cannulated insertion tool 138 keep the ends 126 within the bore 128 and distal of the ports 134 while allowing rotation of the suture receiver 122 within the bore 128.

The invention now being fully described, it will be apparent to one of ordinary skill in the art that many modifications and changes can be made thereto without departing from the spirit or scope of the invention as defined in the following claims.

What is claimed is:

1. A suture anchor comprising:
an anchor body comprising a longitudinal axis and bone engaging external threads oriented for rotation about the longitudinal axis; and
a suture attachment on the anchor body rotational with respect to the body about the longitudinal axis whereby to help reduce twisting of one or more sutures which may be attached to the suture attachment as the anchor body is threaded into a bone.

2. The suture anchor of claim 1 wherein the body and suture attachment are sterile and packaged in a bacteria proof enclosure.

3. The suture anchor of claim 1 wherein at least one suture is attached to the suture attachment.

4. The suture anchor of claim 3 wherein the at least one suture comprises a loop of suture affixed to the suture attachment, the loop having ends extending outwardly from the suture anchor body and suture attachment.

5. The suture anchor of claim 3 wherein the at least one suture comprises a plurality of suture loops of suture affixed to the suture attachment, each loop having ends extending outwardly from the suture anchor body and suture attachment.

6. The suture anchor of claim 1 and further comprising an axial bore entering the body from a proximal end thereof and wherein the suture attachment is located on the body such that one or more sutures attached thereto can extend out of the axial bore at the body proximal end.

7. The suture anchor of claim 6 wherein the body comprises a first abutment surface within the axial bore and wherein the suture attachment comprises an opposing second abutment surface engaged with the first abutment surface such that the suture attachment is rotatable in the axial bore against the first abutment surface and the engagement between the first abutment surface and the second abutment surface prevents the suture attachment from moving proximally out of the axial bore.

8. The suture anchor of claim 7 wherein the first abutment surface and second abutment surface are annular.

9. The suture anchor of claim 6 wherein the suture attachment comprises a disc having a plurality of axial holes therethrough for receiving one or more loops of suture therethrough.

10. The suture anchor of claim 6 wherein the suture attachment comprises a proximal cylindrical portion having one or more radially extending bosses thereon, the cylindrical portion being received within the axial bore with the one or more bosses adjacent a wall of the anchor body forming the axial bore such that suture looped about the one or more bosses is retained by the one or more bosses within the body between the wall and cylindrical portion.

11. A method of attaching tissue to a bone comprising the steps of:
passing a first portion of a suture length through the tissue;
threading a suture anchor having a second portion of the suture length into a bone hole in the bone, the suture anchor comprising an anchor body comprising a longitudinal axis and bone engaging external threads oriented for rotation about the longitudinal axis; and a suture attachment on the anchor body rotational with respect to the body about the longitudinal axis with the second portion of the suture length attached to the suture attachment; and
rotating the suture attachment with respect to the anchor body to reduce twisting of the suture length induced by the step of threading the suture anchor body into the bone hole.

12. The method according to step 11 wherein the second suture portion comprises a loop of suture affixed to the suture attachment and a pair of suture segments extending therefrom and wherein the step of rotating the suture attachment reduces twisting of the pair of suture segment with respect to each other.
